(19) United States
(12) Patent Application Publication
(10) Pub. No.: US 2003/0166696 A1
(43) Pub. Date: Sep. 4, 2003

(54) Pramipexole for the treatment of HIV dementia

(75) Inventors: Ralph Warsinsky, Mainz (DE); Klaus Mendla, Ingelheim (DE); Sandra Fleissner, Ingelheim (DE); Lothar Kussmaul, Bernstadt (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/348,293

(22) Filed: Jan. 21, 2003

Related U.S. Application Data
(60) Provisional application No. 60/386,165, filed on Jun. 5, 2002.

(30) Foreign Application Priority Data
Jan. 24, 2002 (DE) DE 102 03 103.7

(51) Int. Cl. A61K 31/428

(52) U.S. Cl. 514/367

(57) ABSTRACT

The invention relates to the use of pramipexole and the pharmacologically acceptable acid addition salts thereof as well as hydrates and solvates thereof, for preparing a pharmaceutical composition for the prevention and/or treatment of HIV encephalopathy.

### RELATED APPLICATIONS

Benefit of U.S. Provisional Application Ser. No. 60,386,165, filed on Jun. 5, 2002, is hereby claimed.

### FIELD OF THE INVENTION

The invention relates to the use of pramipexole (2-amino-4,5,6,7-tetrahydro-6-n-propylamino-benzothiazole), the (+) or (−) enantiomer thereof, the pharmacologically acceptable acid addition salts thereof as well as hydrates and solvates thereof, for preparing a pharmaceutical composition for the prevention and/or treatment of HIV encephalopathy.

### BACKGROUND TO THE INVENTION

2-Amino-6-n-propylamino-4,5,6,7-tetrahydrobenzo-thiazole is a D2/D3 dopamine receptor agonist which is also known in the art by the name pramipexole or the (+)-enantiomer thereof by the name SND 919. Pramipexole and processes for preparing it are known for example from EP-A-186 087 and U.S. Pat. No. 4,886,812. It is known in particular for the treatment of schizophrenia and particularly for the treatment of Parkinson's disease. In addition, the neuroprotective effect of pramipexole was described in WO 009618395.

HIV infections are frequently associated with neurological functional disorders which take the form of behavioural disorders, motor problems and cognitive impairment (Czub et al., Acta neuropathol., 2001, 101; 85-91). HIV dementia may be mentioned, in particular, as a symptom of these functional disorders.

The treatment of HIV encephalopathy with conventional agents for treating Parkinson's, such as L-dopa or selegiline, has proved ineffective.

The aim of the present invention is to provide an active substance for treating HIV encephalopathy, particularly for treating HIV dementia.

### DESCRIPTION OF THE INVENTION

Surprisingly it has been found that pramipexole is suitable for use in the prevention and/or treatment of HIV encephalopathy, particularly HIV dementia.

Therefore, the present invention relates to the use of pramipexole and the pharmacologically acceptable acid addition salts as well as hydrates and solvates thereof for preparing a pharmaceutical composition for the prevention and/or treatment of HIV encephalopathy.

It is preferable to use the pramipexole (+) enantiomer and the pharmacologically acceptable acid addition salts as well as hydrates and solvates thereof to prepare a pharmaceutical composition for the prevention and/or treatment of HIV encephalopathy.

It is also preferable to use the pramipexole (−) enantiomer and the pharmacologically acceptable acid addition salts as well as hydrates and solvates thereof to prepare a pharmaceutical composition for the prevention and/or treatment of HIV encephalopathy.

Most preferably, pramipexole is used to prepare a pharmaceutical composition for the treatment of HIV dementia.

It is also particularly preferred to use pramipexole to prepare a pharmaceutical composition for the treatment of HIV-associated motor disorders.

Furthermore it is particularly preferred to use pramipexole to prepare a pharmaceutical composition for the treatment of HIV-associated cognitive impairment.

In particular, it is preferred to use pramipexole to prepare a pharmaceutical composition for the treatment of HIV-associated behavioural disorders.

The present invention also relates to the use of pramipexole in conjunction with one or more active substances selected from the group consisting of nucleoside and non-nucleoside inhibitors of reverse transcriptase, HIV protease inhibitors and other HIV replication inhibitors, antiviral active substances within the scope of an HAART (highly active antiretroviral therapy), AIDS vaccines, inhibitors of virus adhesion and virus uptake in mammalian cells, particularly CXCR4 and CCR5 chemokine receptor antagonists, while combinations with a plurality of antiviral active substances within the scope of a Haart and CXCR4 and CCR5 chemokine receptor antagonists are particularly preferred, the CXCR4 and CCR5 chemokine receptor antagonists being most preferred.

The invention further relates to a pharmaceutical composition containing pramipexole in conjunction with one or more active substances selected from the group consisting of nucleoside and non-nucleoside inhibitors of reverse transcriptase, HIV protease inhibitors and other HIV replication inhibitors, antiviral active substances within the scope of a HAART (highly active antiretroviral therapy), AIDS vaccines, inhibitors of virus adhesion and virus uptake in mammalian cells, particularly CXCR4 and CCR5 chemokine receptor antagonists, while combinations with a plurality of antiviral active substances within the scope of a Haart and CXCR4 and CCR5 chemokine receptor antagonists are particularly preferred, the CXCR4 and CCR5 chemokine receptor antagonists being most preferred.

Within the scope of the present invention pramipexole is preferably used to treat HIV-infected patients.

Pramipexole may be used within the scope of the present invention as a racemate, in the form of its (+) or (−) enantiomer. Moreover, pramipexole may be used in the form of the pharmaceutically acceptable acid addition salts thereof as well as optionally in the form of its hydrates and/or solvates. By pharmaceutically acceptable acid addition salts are meant according to the invention the salts selected from the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid, of which the salts of hydrochloric acid, hydrobromic acid, sulphuric acid, phosphoric acid and acetic acid are particularly preferred. The salts of hydrochloric acid are particularly important. Most preferably, within the scope of the present invention, therefore, the hydrochlorides of pramipexole are used, pramipexole dihydrochloride being of particular significance. Of the hydrates of pramipexole, pramipexole dihydrochloride monohydrate is particularly preferred.

For treatment and/or prevention of the medical indications described above, in addition to monotherapy using pramipexole it is also possible, as an alternative, to carry out a combined therapy using pramipexole with one or more pharmaceutically active compounds.

The effect of pramipexole according to the invention will be illustrated by the examples that follow. They serve merely to illustrate the invention and are not intended to be seen as restrictive.

Pramipexole is capable of mopping up various radicals and thereby rendering them harmless. As free radicals play a causative role in the development of HIV encephalopathy, particularly HIV dementia, the use of pramipexole in HIV dementia produces a therapeutic effect. The scavenging function of pramipexole is demonstrated inter alia by the following experimental data in tests A (in vitro), B (in vivo) and C (in vitro):

A. The reduced H₂O₂ formation of isolated mitochondria as in vitro parameters for endogenous radical stress:

Mitochondria reduce 95% of the oxygen to water. As a (patho)physiological secondary reaction, up to 5% of the oxygen is also incompletely reduced to the superoxide anion, which is normally reduced via hydrogen peroxide to water. Overproduction and/or reduced detoxification of superoxide anion and hydrogen peroxide leads to the development of oxidative stress. This is crucially implicated in neuronal cell death in all kinds of neurodegenerative diseases.

In the presence of the energy substrate succinate, isolated mitochondria produce H₂O₂, which results from the dismutation of superoxide anion. In the presence of amplexRed™ and horseradish peroxidase, the fluorogenic chromophor resorufin is formed, which is measured and quantified in a kinetic test. The quantity of H₂O₂ formed by the mitochondria can be increased by the addition of the quinone analogue duroquinone.

The test results are shown in Table 1.

TABLE 1
In vitro mitochondria test.

| | Quantity of H₂O₂ formed |
|---|---|
| Substrate | (%) |
| 5 mM succinate | 100.0 ± 0.8 |
| 5 mM succinate + | 152.2 ± 1.4 |
| 3 μM duroquinone | |
| 5 mM succinate + | 58.3 ± 24.6 |
| 3 μM duroquinone + 100 μM pramipexole | |

Pramipexole reduces the duroquinone-induced formation of H₂O₂ to about 50-60%. As pramipexole neither reacts with H₂O₂ nor influences the detoxification of H₂O₂ by the mitochondrial metabolism, Table 1 shows that the superoxide anion is detoxified by pramipexole. This reduces the quantity of the resultant product H₂O₂ formed from O₂⁻.

B The aconitase activity as an ex vivo parameter for endogenous radical stress:

Aconitase is a mitochondrial enzyme which catalyses the conversion of citrate into isocitrate in the citrate cycle. In its active centre it has an iron-sulphur cluster [4Fe-4S] which is needed for the catalytic activity. This iron-sulphur cluster is specifically destroyed by the radicals superoxide anion (O₂⁻) and nitrogen monoxide (NO) [3Fe-4S] (Gardner et al, 1995, Hausladen and Fridovich, 1996, Longo et al., 2000). In this way the enzyme is inactivated.

Mice (C57BL6) were treated with pramipexole by oral route for 4 days in a dose of 2×1 mg/kg KG/d. The animals were then anaesthetised, killed, their brains were removed and the mitochondria (location of the enzyme and site of the radical formation) were isolated. Then the activity was determined by means of a coupled optical test. In addition, the protein quantity of the aconitase was determined by immunoblot analyses.

The animals which were treated with pramipexole have a higher aconitase activity (153±16%) than the control animals (treated with 0.9% saline; 100±8%; n=4). As the amount of protein in the aconitase was the same in both groups (Ctrl: 100±14% as against 103±27% in the pramipexole group) and consequently a different expression was ruled out, the increased activity of the aconitase in the animals treated with pramipexole is evidence of a reduced radical stress in the animals (Gardner P. R. Raineri I., Epstein L. B. and White C. W. (1995) Superoxide Radical and Iron modulate Aconitase activity in mammalian cells, J. Bio. Chem. 270, 13399-13405; Hausladen A. and Fridovich J. (1996) Measuring Nitric Oxide and Superoxide: Rate Constants for Aconitase Reactivity, Meth. Enzym. 269, 37-41; Longo V. D., Viola K. L., Klein W. L., Finch C. E. (2000) Reversible Inactivation of superoxide-sensitive aconitase in Aβ1-42 treated neuronal cells, J. Neurochem. 75, 1977-1985).

C The formation of fluorescein-2-triazole as a detection system for the scavenging function of NO by pramipexole.

If nitrogen monoxide (NO) is produced in large amounts in the body (e.g. in inflammatory processes), the molecule as a result of its high reactivity also contributes substantially to the development of oxidative stress which eventually leads to the death of the cell.

Using various NO donors, NO can be formed in situ. The donors differ in their half-life, i.e. at the same concentration they release different amounts of NO per unit of time. In a protein- or cell-free system the NO generation was measured by means of the formation of triazole from diaminofluorescein and an NO donor. Different concentrations of pramipexole or (+) enantiomer were added to this system. Inhibition of the formation of triazole was observed. The IC values are in the range from 13 μM-80 μM depending on the donor put in and the buffer/medium used. Thus, pramipexole and the (+) enantiomer act as NO scavengers.

The dosage of pramipexole naturally depends to a great extent on the clinical picture. For example, without restricting the present invention thereto, pramipexole may be used in doses of about 0.05 to 7.5 mg, preferably 0.1 to 5 mg per day. These doses are based on pramipexole in the form of its free base. Based on the salt form pramipexole dihydrochloride monohydrate which is preferably used, the doses mentioned above correspond to about 0.07 to 10.65 mg, preferably 0.14 to 7.1 mg of pramipexole dihydrochloride monohydrate per day.

One possible dosing method, which is to be understood as being merely an illustrative example, is described below, based on pramipexole in the form of its free base: individual dosage titration at weekly intervals depending on activity and tolerance. 1st week: 1 tablet containing 0.088 mg of pramipexole 3 times a day; 2nd week: 1 tablet containing 0.18 mg of pramipexole 3 times a day; 3rd week and thereafter: ½ tablet containing 0.7 mg of pramipexole 3 times a day.

Within the scope of the use according to the invention pramipexole may be administered orally, transdermally, intrathecally, by inhalation or parenterally. Suitable preparations include for example tablets, capsules, suppositories, solutions, syrups, emulsions, dispersible powders or patches. Regarding possible embodiments of a transdermal preparation which may be used according to the invention we now refer to the embodiments described by way of example in U.S. Pat. No. 5,112,842, to which reference is hereby expressly made. Suitable tablets may be produced for example by mixing the active substance or substances with known excipients, for example inert diluents, such as calcium carbonate, calcium phosphate or lactose, disintegrants such as corn starch or alginic acid, binders such as starch or gelatine, lubricants such as magnesium stearate or talc, and/or agents for achieving delayed release such as carboxymethylcellulose, cellulose acetate phthalate, or polyvinyl acetate. The tablets may also consist of several layers.

The following are some examples of pharmaceutical preparations which may be used according to the invention. These are intended solely as an illustration without restricting the subject matter of the invention thereto.

Tablet 1:

| Ingredients: | mg |
|---|---|
| pramipexole dihydrochloride monohydrate | 1.00 |
| mannitol | 121.50 |
| maize starch | 79.85 |
| highly dispersed silicon dioxide, anhydrous | 2.30 |
| Polyvidone K25 | 2.35 |
| magnesium stearate | 3.00 |
| Total | 210.00 |

Tablet 2:

| Ingredients: | mg |
|---|---|
| pramipexole | 0.5 |
| mannitol | 122.0 |
| maize starch. dried | 61.8 |
| maize starch | 18.0 |
| highly dispersed silicon dioxide, anhydrous | 2.4 |
| Polyvidone K25 | 2.3 |
| magnesium stearate | 3.0 |
| Total | 210.0 |

Tablet 3:

| Ingredients: | mg |
|---|---|
| pramipexole | 0.25 |
| mannitol | 61.00 |
| maize starch | 39.90 |
| highly dispersed silicon dioxide, anhydrous | 1.20 |
| Polyvidone K25 | 1.15 |
| magnesium stearate | 1.5 |
| Total | 105.00 |

Tablet 4:

| Ingredients: | mg |
|---|---|
| pramipexole | 0.125 |
| mannitol | 49.455 |
| maize starch dried | 25.010 |
| maize starch | 7.300 |
| highly dispersed silicon dioxide, anhydrous | 0.940 |
| Polyvidone K25 | 0.940 |
| magnesium stearate | 1.230 |
| Total | 85.000 |

Solution for injection:

| | |
|---|---|
| pramipexole dihydrochloride monohydrate | 0.3 mg |
| sodium chloride | 0.8 mg |
| benzalkonium chloride | 0.01 mg |
| water for injections ad 100 ml | |

What is claimed is:

1. A method for treating HIV encephalopathy which comprises administering to an HIV-infected human suffering from HIV encephalopathy a therapeutically effective amount of pramipexole or a pharmacologically acceptable acid addition salt thereof.

2. The method of claim I wherein the (+) enantiomer of pramipexole is employed.

3. The method of claim I wherein the (−) enantiomer of pramipexole is employed.

4. A method for treating an HIV-associated motor disorder which comprises administering to an HIV-infected human suffering from an HIV-associated motor disorder a therapeutically effective amount of pramipexole or a pharmacologically acceptable acid addition salt thereof.

5. The method of claim I wherein the (+) enantiomer of pramipexole is employed.

6. The method of claim I wherein the (−) enantiomer of pramipexole is employed.

7. A method for treating an HIV-associated cognitive impairment which comprises administering to an HIV-infected human suffering from an HIV-associated cognitive impairment a therapeutically effective amount of pramipexole or a pharmacologically acceptable acid addition salt thereof.

8. The method of claim I wherein the (+) enantiomer of pramipexole is employed.

9. The method of claim I wherein the (−) enantiomer of pramipexole is employed.

10. A method for treating an HIV-associated motor disorder which comprises administering to an HIV-infected human suffering from an HIV-associated behavioural disorder a therapeutically effective amount of pramipexole or a pharmacologically acceptable acid addition salt thereof.

11. The method of claim I wherein the (+) enantiomer of pramipexole is employed.

12. The method of claim I wherein the (−) enantiomer of pramipexole is employed.

13. A method for treating HIV infection which comprises administering to an HIV-infected person pramipexole and one or more additional active substances selected from the group consisting of nucleoside and non-nucleoside inhibitors of reverse transcriptase, HIV protease inhibitors and other HIV replication inhibitors, antiviral active substances within the scope of an HAART (highly active antiretroviral therapy), AIDS vaccines and inhibitors of virus adhesion and virus uptake in mammalian cells.
